(19) United States
(12) Patent Application Publication
(10) Pub. No.: US 2026/0335266 A1
(43) Pub. Date: Oct. 29, 2026

(54) COMPOSITIONS TO INCREASE GLUTATHIONE LEVELS AND PROCESSES FOR MAKING THE SAME

(71) Applicant: THE PROIMMUNE COMPANY, LLC, Rhinebeck, NY (US)

(72) Inventors: Albert CRUM, Rhinebeck, NY (US); Alberta CRUM, Rhinebeck, NY (US); Joseph FORTUNAK, Rhinebeck, NY (US)

(21) Appl. No.: 19/109,885

(22) PCT Filed: Sep. 8, 2023
(86) PCT No.: PCT/GB2023/052336

§ 371 Date: Mar. 7, 2025

Related U.S. Application Data
(60) Provisional application No. 63/404,808, filed on Sep. 8, 2022.

(51) Int. Cl. A61K 31/198; A23L 33/16; A23L 33/175; A61K 9/14; A61K 33/04

(52) U.S. Cl. CPC A61K 31/198; A23L 33/16; A23L 33/175; A61K 9/143; A61K 33/04

(57) ABSTRACT

A method for the preparation of a homogeneous nutritional or therapeutic composition comprising glutamine or glutamic acid, cystine, glycine and a selenium source comprises the steps of a) a serial dilution of the selenium source, b) milling and/or sieving of the glutamine or glutamic acid, cystine and glycine individually to give a particle size (D90) of between about 50 microns and about 300 microns, c) mixing the selenium source, glutamine or glutamic acid, cystine and glycine to produce a homogeneous mixture with a particle size (D90) of between about 25 microns and about 300 microns. Compositions made according to the method are also contemplated.

### FIELD OF THE INVENTION

This invention relates to nutritional or therapeutic compositions and processes for making the same useful for treating mammals to increase their body content of glutathione above a pretreatment level thereby to enhance the immune activity of the treated mammal. More specifically, it relates to compositions containing a selenium compound together with a glutathione precursor which is a mixture of glutamic acid, cystine and glycine or glutamine, cystine and glycine and processes to make the same.

### BACKGROUND

Glutathione is a tripeptide and a major reducing agent in the mammalian body. Its chemical structure is:

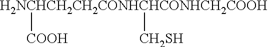

or, more simply GLU-CYS-GLY.

Its chemical name is glutamyl-cysteinyl-glycine. Like many other small peptides in the mammalian body, it is not synthesized by procedures involving DNA, RNA and ribosomes. Rather, it is synthesized from the amino acids available in the body by procedures utilizing enzymes and other body components such as adenosine triphosphate as an energy source. It is generally recognized that many disease processes are attributed to the presence of elevated levels of free radicals, reactive oxygen species (ROS) and reactive nitrogen species (RNS). Such as superoxide, hydrogen peroxide, singlet oxygen, peroxynitrite, hydroxyl radicals, hypochlorous acid (and other hypohalous acids) and nitric oxide.

Mammalian cells have numerous mechanisms to eliminate these damaging free radicals and reactive species. One such mechanism includes the glutathione system, which plays a major role in direct destruction of reactive oxygen compounds and also plays a role in the body's defense against infection. It is known that insufficient levels of glutathione may result in the onset of numerous diseases. Diseases of aging appear to be associated with a drop in glutathione levels. Moreover, since there is no evidence of transport of glutathione into cells, glutathione must be produced intracellularly.

One of the most important contributions of glutathione to mammalian health is its participation in the proper functioning of the immune system to respond to infection or other types of trauma. It is known that weakening of the immune system caused by infection or other traumas occurs concurrently with depletion of glutathione in body tissues. It is known, also, that such weakening can be reversed by replenishing the supply of glutathione. It is believed that glutathione accomplishes its salutary effects by protecting immune cells against the ravages of oxidizing agents and free radicals. There is a need for compositions and methods to aid in elimination of damaging free radicals and reactive oxygen and nitrogen species.

One possible mechanism for achieving this may be through enhancement of glutathione levels in patients utilizing precursors for glutathione synthesis. There is some question as to whether orally ingested glutathione is available to enhance the immune system. Since it is a tripeptide, conventional wisdom suggests that it would be hydrolyzed in the intestinal system to release the free amino acids. Even if some of the tripeptide gets through the gastrointestinal wall intact, it is questionable whether it can be absorbed as such into the individual cell, rather than being synthesized intracellularly. Some experts are of the opinion that glutathione resists hydrolysis when taken orally. In any event, it is generally acknowledged that an increase in tissue and cellular concentrations of glutathione facilitates resistance to infective agents by enhancing the immune system. The mucous membrane is the membrane which lines those body passages which communicate directly or indirectly with the exterior. For purposes of this invention, the important parts of the mucous membrane are those portions which line the oral passage, the nose, the anus and the vagina since the compositions are intended for sublingual, buccal, nasal, anal and or vaginal delivery. Oral delivery by sublingual or buccal routes is much preferred because of its convenience. Such delivery may be, for example, in the form of pills, lozenges and tablets which may be retained in the mouth until dissolved. In rare instances, parenteral delivery may be utilized, but this is normally not necessary.

### BRIEF DESCRIPTION OF INVENTION

Amino acid compositions of glutamine, cystine, and glycine have been demonstrated to increase intracellular levels of glutathione when taken as a nutritional composition. This effect is maximized when the availability of these amino acids is in the ratio of approximately 1.0:0.5:1.0. The use of catalytic amounts of selenium and preferably in the forms of L-selenomethionine or selenocystine is also further beneficial to this use. A method of manufacturing a homogeneous, stable composition for such a purpose is not readily available or apparent, however.

Although selenium is an essential mineral, the recommended daily allowance (RDA) of elemental selenium is very low: 55 micrograms (mg) per day. An intake of selenium of 400 mg or more per day is considered unsafe. Indeed, it has been suggested that a daily intake of elemental selenium exceeding 200 mg/day should be avoided for those with poor glycemic control or at risk of diabetes.

The NHANES III national survey reported that the mean dietary intake of selenium for adults in the US ranges between 100.5 and 158.5 mg/day. It is therefore readily apparent that for the purpose of catalyzing intracellular glutathione synthesis, the desired amount of selenium to be delivered should be sufficient to be effective but should not exceed the RDA. Thus, the target amount of elemental selenium to be delivered to an individual in compositions in the prior art (e.g., in US patent reissue numbers RE 39,374 and RE 42,645, both incorporated by reference in their entirety, it is between approximately 1 mg and 7.5 mg/day).

On the other hand, evidence suggests that the useful enhancement of intracellular glutathione synthesis by the amino acids of the instant invention requires much higher doses of glutamine, cystine, and glycine. The suggested combined daily intake of these amino acids for such a purpose is one or two doses of approximately 1600 milligrams (mg) per day. Thus, the total amount of amino acids to be delivered in a unit dose is between approximately 213,000 and 1,600,000 times the amount of elemental selenium desired to be provided.

A problem to be addressed in the process for manufacturing such a product therefore is a reliable, reproducible means of manufacturing a homogeneous composition containing the desired doses of amino acids and selenium. The difficulty of ensuring the homogeneity of the very low concentration of selenium present in the composition, so that an individual will not exceed the RDA of selenium intake, will be evident to one of skill in the art. Thus, a product must contain very low amounts of selenium distributed evenly throughout the composition. As a matter of reference, Example 2 of US RE 42, 645 E calls for 5 micrograms of selenium methionine to be evenly dispersed in 976 milligrams of amino acids and other ingredients to arrive at the desired composition. This is a nearly 200,000-fold dilution of selenium methionine. The difficulty of assuring the homogeneity of selenium content in such a mixture again is evident to one of skill in the art. For purposes of commercial practicality, this method of assuring the homogeneity of selenium content in the final composition must also be scalable. It is desirable to produce the desired composition in amounts of up to one metric ton or even larger scale, in preparing the desired product.

The importance of stability of the composition will also be evident to one of skill in the art. This is important in terms of the uniformity of selenium content in the composition over time. It is further important in terms of the uniformity and stability of the amino acids contained in the composition over the lifetime of the product. Compounded powders are known to be prone to segregation during mixing and upon storage. Amino acids are also known to react with themselves (dimerization), or with excipients (e.g., the Maillard reaction between amino acid residues and reducing sugars), to yield degradation products over time.

A third problem to be solved in preparing such a pharmaceutical composition is convenience of administration. Large doses of a composition to be administered orally to a human are inconvenient. The likelihood of compliance with a recommended dose for a composition is inversely correlated with the size of the dose. Thus, the amounts of inactive ingredients (commonly called excipients) in a composition are minimized for high-dose materials to enhance compliance with the recommended dose.

### SUMMARY OF THE INVENTION

In a first embodiment a method for the preparation of a homogeneous nutritional or therapeutic composition comprising glutamine, cystine, glycine and a selenium source comprising the steps of
(a) a serial dilution of the selenium source
(b) milling and/or sieving of the glutamine, cystine and glycine individually to give a particle size (D90) of between about 25 (preferably 50) microns and about 300 microns
(c) mixing the selenium source, glutamine, cystine and glycine to produce a homogeneous mixture with a particle size (D90) of between about 50 microns and about 300 microns is provided.

In another embodiment the ratio of glutamine, cystine and glycine in the composition is in a molar ratio of between about 0.9 to about 1.1 in glutamine; between about 0.3 to about 0.6 in cystine and between about 1 to about 2 in glycine.

In another embodiment the selenium source comprises between about 0.0001% and about 1% by weight of the composition.

In another embodiment the selenium source is a non-toxic water soluble organic or inorganic selenium compound.

In another embodiment the selenium source is selected from: sodium selenite, selenium methionine, selenium cysteine, selenium cystine, a mono-seleno amino acid with 6 to 12 carbon atoms in the chain or a di-seleno amino acid with 10 to 24 carbon atoms in the chain, including but not limited to, encapsulated forms of such sources of selenium.

In another embodiment the selenium source is Selenium Select 5000 (Sabinsa Corporation).

In another embodiment the molar ratio in the composition is about 1 in glutamine, about 0.5 in cystine and about 1 in glycine.

In another embodiment the particle size (D90) of the homogeneous mixture is between about 100 microns and about 200 microns.

In another embodiment the particle size of the homogeneous mixture is measured by a particle size analyzer and represents the volume-weighted distribution of the particles.

In an embodiment a method for the preparation of a homogeneous nutritional or therapeutic composition comprising glutamic acid, cystine, glycine and a selenium source comprising the steps of
(a) a serial dilution of the selenium source
(b) milling and/or sieving of the glutamic acid, cystine and glycine individually to give a particle size (D90) of between about 50 microns and about 300 microns (c) mixing the selenium source, glutamic acid, cystine and glycine to produce a homogeneous mixture with a particle size (D90) of between about 25 (preferably 50) microns and about 300 microns is provided.

In another embodiment the ratio of glutamic acid, cystine and glycine in the composition is in a molar ratio of between about 0.9 to about 1.1 in glutamic acid; between about 0.3 to about 0.6 in cystine and between about 1 to about 2 in glycine.

In another embodiment the selenium source comprises between about 0.0001% and about 1% by weight of the composition, such that the selenium source provides between about 4 micrograms and about 55 micrograms of selenium per daily dose.

In another embodiment the selenium source is a non-toxic, water soluble, organic or inorganic selenium compound

In another embodiment the selenium source is sodium selenite, selenium methionine, selenium cystine, a monoseleno amino acid with 6 to 12 carbon atoms in the chain or a di-seleno amino acid with 8 to 24 carbon atoms in the chain.

In another embodiment the selenium source is Selenium Select 5000.

In another embodiment the molar ratio in the composition is about 1 in glutamine, about 0.5 in cystine and about 1 in glycine.

In another embodiment the particle size (D90) of the homogeneous mixture is between about 100 microns and about 200 microns.

In another embodiment the particle size of the homogeneous mixture is measured by a particle size analyzer and represents a volume-weighted distribution.

In another embodiment a pharmaceutical composition prepared according to the methods above, wherein the particle size of the homogeneous mixture (D90) is between about 25 (preferably 50) microns and about 300 microns.

In another embodiment a pharmaceutical composition prepared according to the methods above, wherein the composition is stable for at least 24 months under normal storage conditions.

### DETAILED DESCRIPTION OF THE INVENTION

When describing the compounds and processes of the invention, the terms used are to be construed in accordance with the following definitions, unless a context dictates otherwise.

The term “about” as used herein when referring to a measurable value such as a parameter, a purity, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The manufacturing batch examples cited below provide approximate quantities to be charged in the manufacture of a single, 1,000 kg batch of product and as represented in an illustrative master batch record. These approximate quantities are not meant to be in any way limiting to the actual relative content charged of amino acids or selenium source, provided that such amounts are within the safe recommended daily dose of ingredients used as dietary supplements (e.g., selenium) and determined by the USDA.

### EXAMPLES

The manufacturing batch examples cited below provide approximate quantities to be charged in the manufacture of product on a scale of approximately 900 kg-1,000 kg per batch. These examples are illustrative of the manufacturing directions in a master batch record and of actual manufactured lots. These approximate quantities are not meant to be in any way limiting to the actual relative content charged of amino acids or selenium source, provided that such amounts are within the safe recommended daily dose of ingredients used as dietary supplements (e.g., selenium) and determined by the USDA.

### Example One. Preparation of a Selenium Containing Pre-Blend

A sample of dibasic calcium phosphate (200.0 gm, Sigma Aldrich, US Pharmacopeial grade) is passed through a 40-mesh sieve and visually inspected to ensure that the material has completely passed the sieve. A sample of approximately 2.53 g of L-selenomethionine (Calbiochem) is separately weighed. Approximately one hundred grams of the calcium phosphate is charged to a benchtop blender with a nominal volume of one liter. The blender is further charged with approximately 1.25 g of L-selenomethionine. The sample is blended for a minimum of approximately ten minutes. Blended is temporarily stopped, and the remaining charge of L-selenomethionine is added. The mixture is further blended for an additional minimum time of approximately ten minutes. Blended is again temporarily stopped, and the remaining approximately 100 grams of dibasic calcium phosphate is added to the blender. Blending is resumed for a minimum of approximately twenty minutes. The mixture is discharged from the blender and is stored appropriately for further use. A sample of the blend may be tested by an appropriate method (e.g., atomic absorption) to ensure that the blend contains a uniform content of approximately 5,000 ppm (parts-per-million) of elemental selenium.

Note: The selenium source of Example One and as used in subsequent examples may be optionally in the form of organic selenium (e.g. L-selenomethionine, selenocysteine, selenocystine, Se-Methyl-selenocystine), or inorganic selenium (e.g., sodium or potassium selenite or selenate).

### Example Two. Preparation of the Amino Acid Composition

A sample of 1.013 kg of Selenium Select 5000™ (Sabinsa Corporation, containing 5,000 ppm of elemental Selenium) is weighed into a clean, dry container. L-Glutamine (344.4 kg; 2,357 moles), glycine (342.6 kg; 4,564 moles), and L-cystine (212.0 kg; 882 moles) are separately weighed and held in containers for subsequent use. Approximately 10 kilograms each of L-glutamine, L-cystine, and glycine are separately weighed and charged through a 16-mesh sieve into a container. The full desired amount (1.013 kg) of Selenium Select 5000 is similarly charged through a 16-mesh sieve into the container. Approximately 10 kilograms each of L-glutamine, L-cystine, and glycine are again separately weighed and charged through a 16-mesh sieve into the same container. The contents of the container are transferred to a clean, twin-cone blender and are blended for a minimum of approximately five minutes. This blended material is discharged into an appropriate container for subsequent use. All of the remaining L-glutamine, L-cystine, and glycine are passed through a 16-mesh sieve into appropriate and separate containers. Approximately half of the L-glutamine, L-cystine, and glycine are now charged to the blender. The blended mixture of amino acids containing the Selenium Select 5000 is subsequently charged to the blender. This is followed by addition of the remaining amino acids. The mixture of powders is then blended for a minimum of approximately 20 minutes. The blended material is discharged from the blender and held in appropriate containers for subsequent use.

### Example Three. Preparation of the Amino Acid Composition

A sample of 0.99 kg of Selenium Select 5000™ (Sabinsa Corporation, containing 5,000 ppm of elemental Selenium) is weighed into a clean, dry container. L-Glutamine (386 kg; 2,640 moles), glycine (198 kg; 2,640 moles), and L-cystine (317.2 kg; 1,320 moles) are separately weighed and held in containers for subsequent use. Approximately 10 kilograms each of L-glutamine, L-cystine, and glycine are separately weighed and charged through a 16-mesh sieve into a container. The full desired amount (0.99 kg) of Selenium Select 5000 is similarly charged through a 16-mesh sieve into the container. Approximately 10 kilograms each of L-glutamine, L-cystine, and glycine are again separately weighed and charged through a 16-mesh sieve into the same container. The contents of the container are transferred to a clean, twin-cone blender and are blended for a minimum of approximately five minutes. This blended material is discharged into an appropriate container for subsequent use. All of the remaining L-glutamine, L-cystine, and glycine are passed through a 16-mesh sieve into appropriate and separate containers. Approximately half of the L-glutamine, L-cystine, and glycine are now charged to the blender. The blended mixture of amino acids containing the Selenium Select 5000 is subsequently charged to the blender. This is followed by addition of the remaining amino acids. The mixture of powders is then blended for a minimum of approximately 20 minutes. The blended material is discharged from the blender and held in appropriate containers for subsequent use.

### Example Four. Preparation of the Amino Acid Composition

A sample of approximately 1.01 kg of L-selenomethionine in dicalcium phosphate prepared as in Example 1 and containing approximately 5,000 ppm of elemental Selenium, is weighed into a clean, dry container. L-Glutamine (344.4 kg; 2,357 moles), glycine (342.6 kg; 4,564 moles), and L-cystine (212.0 kg; 882 moles) are separately weighed and held in containers for subsequent use. Approximately 10 kilograms each of L-glutamine, L-cystine, and glycine are separately weighed and charged through a 16-mesh sieve into a container. The full desired amount (1.01 kg) of L-selenomethionine/dicalcium phosphate blend is similarly charged through a 16-mesh sieve into the container. Approximately 10 kilograms each of L-glutamine, L-cystine, and glycine are again separately weighed and charged through a 16-mesh sieve into the same container. The contents of the container are transferred to a clean, twin-cone blender and are blended for a minimum of approximately five minutes. This blended material is discharged into an appropriate container for subsequent use. All of the remaining L-glutamine, L-cystine, and glycine are passed through a 16-mesh sieve into appropriate and separate containers. Approximately half of the L-glutamine, L-cystine, and glycine are now charged to the blender. The blended mixture of amino acids containing the Selenium pre-blend is subsequently charged to the blender. This is followed by addition of the remaining amino acids. The mixture of powders is then blended for a minimum of approximately 20 minutes. The blended material is discharged from the blender and held in appropriate containers for subsequent use.

### Example Five. Preparation of the Amino Acid Composition

A sample of 1.11 kg of L-selenocystine in dicalcium phosphate prepared as in Example 1 and containing approximately 5,000 ppm of elemental Selenium, is weighed into a clean, dry container. L-Glutamine (428 kg; 2,929 moles), glycine (220 kg; 2,930 moles), and L-cystine (352 kg; 1,465 moles) are separately weighed and held in containers for subsequent use. Approximately 10 kilograms each of L-glutamine, L-cystine, and glycine are separately weighed and charged through a 16-mesh sieve into a container. The full desired amount (1.11 kg) of L-selenocystine in dicalcium phosphate is similarly charged through a 16-mesh sieve into the container. Approximately 10 kilograms each of L-glutamine, L-cystine, and glycine are again separately weighed and charged through a 16-mesh sieve into the same container.

The contents of the container are transferred to a clean, twin-cone blender and are blended for a minimum of approximately five minutes. This blended material is discharged into an appropriate container for subsequent use. All of the remaining L-glutamine, L-cystine, and glycine are passed through a 16-mesh sieve into appropriate and separate containers. Approximately half of the L-glutamine, L-cystine, and glycine are now charged to the blender. The blended mixture of amino acids containing the Selenium pre-blend is subsequently charged to the blender. The remainder of the mixture of amino acids is then added to the blender. The material is then blended for a minimum of approximately 20 minutes. The blended material is discharged from the blender and held in appropriate containers for subsequent use.

### Example Six. Preparation of the Amino Acid Composition

A sample of approximately 1.11 kg of L-selenocystine in dicalcium phosphate prepared as in Example 1 and containing approximately 5,000 ppm of elemental Selenium, is weighed into a clean, dry container. L-Glutamine (428 kg; 2,929 moles), glycine (220 kg; 2,930 moles), and L-cystine (352 kg; 1,465 moles) are separately weighed and held in containers for subsequent use. Approximately 10 kilograms each of L-glutamine, L-cystine, and glycine are separately weighed and charged through a 16-mesh sieve into a container. The full desired amount (1.11 kg) of L-selenocystine is similarly charged through a 16-mesh sieve into the container. Approximately 10 kilograms each of L-glutamine, L-cystine, and glycine are again separately weighed and charged through a 16-mesh sieve into the same container. The contents of the container are transferred to a clean, twin-cone blender and are blended for a minimum of approximately five minutes. This blended material is discharged into an appropriate container for subsequent use. All of the remaining L-glutamine, L-cystine, and glycine are passed through a 16-mesh sieve into appropriate and separate containers. Approximately half of the L-glutamine, L-cystine, and glycine are now charged to the blender. The blended mixture of amino acids containing the L-selenocystine in dicalcium phosphate is subsequently charged to the blender. The mixture of powders is then blended for a minimum of approximately 20 minutes. The remainder of the L-glutamine, L-cystine, and glycine are now charged to the blender. The mixture of powders is then blended for a minimum of approximately 20 minutes. The blended material is discharged from the blender and held in appropriate containers for subsequent use.

### Example Seven. Preparation of the Amino Acid Composition Containing Magnesium Ascorbate

A sample of approximately 1.11 kg of Selenium Select 5000™ (Sabinsa Corporation, containing 5,000 ppm of elemental Selenium) is weighed into a clean, dry container. L-Glutamine (428 kg; 2,929 moles), glycine (220 kg; 2,930 moles), L-cystine (352 kg; 1,465 moles), and magnesium ascorbate (307 kg; 820 moles) are separately weighed and held in containers for subsequent use. Approximately 10 kilograms each of L-glutamine, L-cystine, glycine, and magnesium ascorbate are separately weighed and charged through a 16-mesh sieve into a container. The full desired amount (1.013 kg) of Selenium Select 5000™ is similarly charged through a 16-mesh sieve into the same container. Approximately 10 kilograms each of L-glutamine, L-cystine, glycine, and magnesium ascorbate are again separately weighed and charged through a 16-mesh sieve into the same container. The contents of the container are transferred to a clean, twin-cone blender and are blended for a minimum of approximately five minutes. This blended material is discharged into an appropriate container for subsequent use. The remaining L-glutamine, L-cystine, and glycine are passed through a 16-mesh sieve into appropriate and separate containers. Approximately half of the L-glutamine, L-cystine, and glycine are now charged to the blender. The blended mixture of amino acids and magnesium ascorbate containing the Selenium Select 5000 is subsequently charged to the blender. This is followed by addition of the remaining amino acids and magnesium ascorbate. The mixture of powders is then blended for a minimum of approximately 40 minutes. The blended material is discharged from the blender and held in appropriate containers for subsequent use.

It will be appreciated by one of skill in the art that these examples are not limiting in any way as to the batch size, molar ratios of L-glutamine, glycine, and L-cystine, and amount and source of selenium used in product manufacturing. One of skill in the art will also appreciate that the exact proportions of selenium source and dicalcium phosphate may be varied according to the need for selenium content in the product. The exact proportions of amino acids and magnesium ascorbate used in the pre-blend may also be varied in relative amounts as desired, so long as the final product is homogeneous in the content of various ingredients used during manufacturing.

The samples used for testing described in Examples 8 and 9 were prepared following the process as described in Example 2.

### Example Eight. Stability of the Composition, Selenium

The product may be sampled from its appropriate containers and tested to establish the stability and content uniformity over time. For instance, samples may be removed and tested for selenium content at appropriate times during storage. Such testing may be carried out by an appropriate method such as ICP-MS (inductively coupled plasma Mass Spectrometry) or AA (Atomic Absorption). Such testing shows that the selenium content determined by such methods matches the theoretical amount of selenium as added during the product manufacture at t=0.

Testing further shows that the selenium content remains at essentially constant levels after testing at 12 months and 24 months after manufacturing and packaging. An illustrative batch of product was calculated to contain 5.65 micrograms per gram (i.e., 5.65 ppm) of elemental selenium in the form of L-selenomethionine at the time of manufacture. Testing at the time of manufacturing using High Performance Liquid Chromatography coupled with ICP-MS indicated a selenium content of 5.58 ppm. Samples of the product were removed at the time points of t=12 months and t=24 months and tested for the presence of selenium. The selenium content in each of the samples tested was found to be both uniform across samples taken from the same batch at the same time, and stable with respect to samples taken at different times of storage. All samples tested were within the limits expected in consideration of the Relative Standard Deviation of the test method and in consideration of expected variability of content uniformity. The sample results for selenium were within +5% for all samples tested. The results obtained at 12 months from samples tested using HPLC coupled with ICP-MS averaged 5.78 ppm (102.3% of expected value). Further, the results of sample testing indicated an average value of 5.58 ppm (98.8% of expected value) at 24 months when tested using Atomic Absorption (AA). These results indicate that the product is stable and homogeneous for selenium content.

### Example Nine. Stability of the Composition, Amino Acids Content

The product was also tested for amino acid content at t=0, and at t=12 months and 24 months. Such testing may be conducted by an appropriate means such as High-Performance Liquid Chromatography (HPLC) with pre- or post-column visualization of the eluted amino acids. The samples may be tested using, for example, official methods in the US and International Pharmacopeia for the determination of amino acids as specifically discussed in the relevant sections of these Pharmacopeia.

Testing using the standard method AOAC 999.13 issued by the Association of Official Agricultural Chemists (AOAC International) can also be used with post-column derivatization to achieve equivalent and satisfactory results. The Relative Standard Deviation for such methods using pre- or post-column derivatization is approximately ±6%-8%, depending upon the amino acid analyzed. Thus, testing of fifteen different samples of product for amino acid analysis after 24 months of storage gave results as shown in Table 1. These results indicate that the product is stable with respect to amino acids content for at least 24 months.

TABLE 1
Test results using High Performance Liquid Chromatography.
Method AOAC 999.13 was used for testing.

| Sample | | | | Glutamic | | | Total |
|---|---|---|---|---|---|---|---|
| ID | Alanine | Histidine | Cystine | Acid | Glycine | Glutamine | (g/100 g) |
| 2327693 | 0.11 | 0 | 20.59 | 0.53 | 40.95 | 39.8 | 101.98 |
| 2327694 | 0.11 | 0 | 20 | 0.54 | 40.83 | 36 | 97.48 |
| 2327695 | 0.11 | 0 | 20.16 | 0.51 | 42.21 | 40.4 | 103.39 |
| 2327696 | 0.14 | 0 | 19.58 | 0.32 | 41.84 | 40.4 | 102.28 |
| 2327697 | 0.11 | 0 | 20.14 | 0.53 | 43.2 | 39.7 | 103.68 |
| 2327698 | 0.1 | 0 | 20.65 | 0.47 | 40.38 | 39.1 | 100.7 |
| 2327699 | 0.19 | 0 | 20.1 | 0.28 | 39.05 | 39.7 | 99.32 |
| 2327700 | 0.12 | 0 | 19.59 | 0.6 | 38.9 | 41.7 | 100.91 |
| 2327701 | 0.19 | 0 | 20.69 | 0.28 | 40.78 | 39.7 | 101.64 |
| 2327702 | 0.16 | 0 | 20.05 | 0.28 | 42.79 | 41.7 | 104.98 |
| 2327703 | 0.16 | 0.1 | 20.61 | 0.33 | 41.65 | 42.4 | 105.25 |
| 2327704 | 0.09 | 0.09 | 19.94 | 0.52 | 40.35 | 38.9 | 99.89 |
| 2327705 | 0.12 | 0.09 | 19.92 | 0.62 | 38.89 | 38.4 | 98.04 |
| 2327706 | 0.11 | 0.15 | 20.29 | 0.5 | 42.68 | 40.1 | 103.83 |
| 2327707 | 0.17 | 0 | 20.91 | 0.32 | 42.12 | 38.4 | 101.92 |

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosures in U.S. patent application No. 63/404,808, from which this application claims priority, and in the abstract accompanying this application are incorporated herein by reference.

1. A method for the preparation of a homogeneous nutritional or therapeutic composition comprising glutamine, cystine, glycine and a selenium source comprising the steps of:
a) a serial dilution of the selenium source;
b) milling and/or sieving of the glutamine, cystine and glycine individually to give a particle size (D90) of greater than or equal to 50 microns and less than or equal to 300 microns; and
c) mixing the selenium source, glutamine, cystine and glycine to produce a homogeneous mixture with a particle size (D90) of greater than or equal to 25 microns and less than or equal to 300 microns.

2. The method according to claim 1, wherein a ratio of glutamine, cystine and glycine in the composition is in a molar ratio of greater than or equal to 0.9 and less than or equal to 1.1 in glutamine; greater than or equal to 0.3 and less than or equal to 0.6 in cystine; and greater than or equal to 1 and less than or equal to 2 in glycine.

3. The method according to claim 1, wherein the selenium source comprises greater than or equal to 0.0001% and less than or equal to 1% by weight of the composition.

4. The method according to claim 1, wherein the selenium source is a non-toxic water soluble organic or inorganic selenium compound.

5. The method according to claim 1, wherein the selenium source is selected from: sodium selenite, selenium methionine, selenium cysteine, selenium cystine, a mono-seleno amino acid with 6 to 12 carbon atoms in the chain or a di-seleno amino acid with 10 to 24 carbon atoms in the chain, including but not limited to, encapsulated forms of such sources of selenium.

6. The method according to claim 1, wherein the selenium source is Selenium Select 5000.

7. The method according to claim 1, wherein the molar ratio in the composition is about 1 in glutamine, about 0.5 in cystine and about 1 in glycine.

8. The method according to claim 1, wherein the particle size (D90) of the homogeneous mixture is greater than or equal to 100 microns and less than or equal to 200 microns.

9. The method according to claim 1, wherein the particle size of the homogeneous mixture is measured by a particle size analyzer and represents the volume weighted distribution of the particles.

10. A method for the preparation of a homogeneous nutritional or therapeutic composition comprising glutamic acid, cystine, glycine and a selenium source comprising the steps of:
a) a serial dilution of the selenium source;
b) milling and/or sieving of the glutamic acid, cystine and glycine individually to give a particle size (D90) of greater than or equal to 50 microns and less than or equal to 300 microns; and
c) mixing the selenium source, glutamic acid, cystine and glycine to produce a homogeneous mixture with a particle size (D90) of greater than or equal to 25 microns and less than or equal to 300 microns.

11. The method according to claim 10, wherein a ratio of glutamic acid, cystine and glycine in the composition is in a molar ratio of greater than or equal to 0.9 and less than or equal to 1.1 in glutamic acid; greater than or equal to 0.3 and less than or equal to 0.6 in cystine; and greater than or equal to 1 and less than or equal to 2 in glycine.

12. The method according to claim 10, wherein the selenium source comprises greater than or equal to 0.0001% and less than or equal to 1% by weight of the composition, such that the selenium source provides greater than or equal to 4 micrograms and less than or equal to 55 micrograms of selenium per daily dose.

13. The method according to claim 10, wherein the selenium source is a non-toxic, water soluble, organic or inorganic selenium compound.

14. The method according to claim 10, wherein the selenium source is sodium selenite, selenium methionine, selenium cystine, a monoseleno amino acid with 6 to 12 carbon atoms in the chain or a di-seleno amino acid with 8 to 24 carbon atoms in the chain.

15. The method according to claim 10, wherein the selenium source is Selenium Select 5000.

16. The method according to claim 10, wherein the molar ratio in the composition is about 1 in glutamine, about 0.5 in cystine, and about 1 in glycine.

17. The method according to claim 10, wherein the particle size (D90) of the homogeneous mixture is greater than or equal to 100 microns and less than or equal to 200 microns.

18. The method according to claim 10, wherein the particle size of the homogeneous mixture is measured by a particle size analyzer and represents a volume-weighted distribution.

19. A pharmaceutical composition prepared according to the method of claim 1, wherein at least one of:
the particle size of the homogeneous mixture (D90) is greater than or equal to 25 microns and less than or equal to 300 microns;
the composition is stable for at least 24 months under normal storage conditions;
or both.

20. (canceled)

21. A pharmaceutical composition prepared according to the method of claim 1, wherein at least one of:
the particle size of the homogeneous mixture (D90) is greater than or equal to 25 microns and less than or equal to 300 microns;
the composition is stable for at least 24 months under normal storage conditions;
or both.
